(19) United States
(12) Patent Application Publication
(10) Pub. No.: US 2025/0057540 A1
(43) Pub. Date: Feb. 20, 2025

(54) EMBOLIC COIL, SYSTEM, AND METHOD FOR MAKING MULTIPLE SELF-ADAPTIVE LOOPS WITH IMPROVED ANCHORING AND FILLING

(71) Applicant: NDI Medical Technologies Inc., Nisantasi-Istanbul (TR)

(72) Inventors: Sadik Semih DEMIRALP, Buffalo, MN (US); Michael Joseph KUSKE, Buffalo, MN (US); Civan ISLAK, Istanbul (TR)

(73) Assignee: Ndi Tip Teknolojileri Anonim Sirket, Nisantasi-Istanbul (TR)

(21) Appl. No.: 18/452,513

(22) Filed: Aug. 18, 2023

(51) Int. Cl. A61B 17/12

(52) U.S. Cl. CPC A61B 17/1214; A61B 17/12113; A61B 2017/00778; A61B 2017/00867; A61B 2017/00964

(57) ABSTRACT

An embolic coil with improved anchor, system, and method of manufacture, formed from shape memory wire comprises primary, secondary, and tertiary coils. Secondary coils may be formed from one or more open, single-turn, multi-turn, or transition loops. Anchor groups at distal and/or proximal portions of the coil may comprise one or more open, single-turn, or multi-turn loops, and may be used to facilitate manufacturing to improve safe anchoring of the coil within an aneurysm sac, regardless of the neck size. The coil may be formed on a mandrel with a groove in one or more spherical and cylindrical surfaces, which may be scaled to fabricate a variety of coil sizes. The multiple coils of the anchor and intermediate portions deploy self-adaptively in separate, non-parallel planes, substantially assuming the inner surface of an irregularly shaped, constricted, or un-constricted, aneurysm and provides improved framing, filling, and uniform fill distribution.

### FIELD OF THE INVENTION

The present invention relates to an embolic coil, system and method and, more particularly, to an embolic coil comprising multiple curves of multiple radii and self-adaptive loops and an improved anchor system located at one or more ends thereof which, in a relaxed condition, deploy multiple loops oriented in separate, non-parallel planes thereby substantially assuming the shape of the inner surface of a vessel at the site of an embolization procedure.

### BACKGROUND OF THE INVENTION

Conventional embolic coils are useful to repair arterial aneurysms or other vascular segments in the surgical treatment thereof. Conventional embolic coil designs and systems provide separate framing and filling and finishing coils to be disposed in the aneurysm sac. Typically, framing coils are selected to be at or less than the diameter of the aneurysm sac, while subsequent filling coils are disposed adjacent and around the framing coil(s) so as to fully fill the aneurysm sac and promote a blood clot. Nonetheless, conventional embolic coils are useful to repair the aneurysm sac of the aneurysm or other adjacent vascular segments in the surgical treatment thereof.

Problems occur upon insertion of the coil when the coil does not anchor to the wall of the aneurysm sac. Anchoring is further difficult in bifurcated aneurysms where the aneurysm is located at the branch point of an artery. Such bifurcated aneurysms are difficult to treat because they have a shape wherein the opening of the aneurysm sac is larger than the body portion of the sac. In such a case, conventional coils may not remain in the sac. Consequently, there is a need for improved anchoring when depositing an embolic coil in the aneurysm sac so as to positively anchor to the wall and secure the embolic coil for further coil distribution to fill the sac in a more uniform way.

Conventional coils may not engage all surfaces of the wall when deposited in the aneurysm sac. Conventional coils can be observed in a fluoroscope to accumulate along a meridian or otherwise form groups of coils at an equatorial region of the aneurysm sac thereby creating voids open to blood flow that may continue to enter the sac, albeit at a diminished flow. Factors contributing to such disadvantages in the art include coil materials, coil length, the diameter of the coil loops, and other design choices. Consequently, there is a need to reduce or eliminate such equatorial region accumulation when depositing an embolic coil in an aneurysm sac. What is needed is an embolic coil that seeks out irregular walls and empty spaces, evenly distributing itself within the sac, especially when it is of irregular shape.

Coil accumulation in one area may create undesirable, additional pressure on the aneurysm sac. Uneven coil distribution is due in part to normal irregularities in the wall of the aneurysm whereby each sac is not spherical and may have numerous bulbus protrusions therein. It has been observed that conventional embolic coils may leave voids in the sac when no more coils can be used in the procedure. Such voids in the group of framing, filling and finishing coils are observed to coalesce in one area of the sac so as to create a void large enough to allow blood flow to continue to enter the sac, albeit in a smaller quantity. Consequently, there is a need to solve such sac-coil distribution problems with an embolic coil that seeks out empty spaces in the aneurysm sac so as to uniformly distribute and fill the irregular shape of each embolic sac.

Each particular aneurysm sac has limit as to the number of coils that can be inserted safely, beyond which no further framing, filling and/or finishing coils may be inserted. A coil fill factor is used to measure the effectiveness of each added coil. Additional coils, in order to pack and support the wall of the aneurysm, increase surgical procedure costs. Conventional embolic coils have a fill factor of about 25% and may be susceptible to the accumulation problem or otherwise may not support all surfaces of the inner wall of the aneurysm. Additionally, coil removal is not a safe option once coil accumulation occurs due to risk of trauma to the aneurysm. As a result, there also is a need for an embolic coil with a high fill factor to reduce costs and improve patient outcomes.

In summary, there is a need for an embolic coil with improved anchoring that also preferentially fills empty spaces so as to uniformly distribute and completely fill irregularly- or open-shaped aneurysms.

### SUMMARY OF THE INVENTION

It is an object of the present disclosure to provide an embolic coil with an improved anchor system having multiple loops oriented in separate, non-parallel planes.

It is an object of the present disclosure to provide an embolic coil that fills in a more uniform way the irregular shape of an aneurysm sac by seeking out empty spaces.

It is an object of the present disclosure to provide an embolic coil that improves the fill factor for an aneurysm at a reduced cost.

Other desirable features and characteristics will become apparent from the subsequent detailed description, the drawings, and the appended claims, when considered in view of this background.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present disclosure, reference will be made to the following Detailed Description, which is to be read in association with the accompanying drawings, which are incorporated in and constitute a part of this specification, show certain aspects of the subject matter disclosed herein and, together with the description, help explain some of the principles associated with the disclosed implementations, wherein:

FIG. 1A is a front, top, right perspective view illustrating a conventional coil disposed in the aneurysm sac, according to the prior art;

FIG. 1B is a top view, taken along an equatorial plane (line EP-EP) of conventional coil disposed in the aneurysm sac shown in FIG. 1A, according to the prior art;

FIG. 2A is a side perspective view illustrating a conventional coil disposed in an aneurysm sac having an unconstrained opening, according to the prior art;

FIG. 2B is a top perspective view, taken along a near normal to an equatorial plane (line EP-EP of FIG. 2A) of a conventional coil disposed in an aneurysm sac, according to the prior art;

FIG. 3A is a front, top perspective view illustrating an embolic coil with distal anchor group, according to an embodiment of the present invention;

FIG. 3B is a front, top, right perspective view illustrating a mandrel configured to manufacture an embolic coil with distal anchor group, according to an embodiment of the present invention;

FIG. 4 is a plan view illustrating a flattened embolic coil overlaid on a reference mandrel blank, according to an embodiment of the present invention;

FIG. 5A is a detail perspective view illustrating the distal anchor portion of an embolic coil, according to an embodiment of the present invention;

FIG. 5B is a detail perspective view illustrating the mandrel portion corresponding to the distal anchor group of an embolic coil, according to an embodiment of the present invention;

FIG. 6A is a detail front view illustrating the multi-turn loop section 6A of FIG. 5A of an embolic coil, according to an embodiment of the present invention;

FIG. 6B is a detail bottom view illustrating the multi-turn loop section 6A of FIG. 5A of an embolic coil, according to an embodiment of the present invention;

FIG. 7A is a front, top, right perspective view illustrating an embolic coil with distal anchor group, according to an alternate embodiment of the present invention;

FIG. 7B is a front, top, right perspective view illustrating a mandrel configured to manufacture an embolic coil with distal anchor group according to an alternate embodiment of the present invention;

FIG. 8 is a plan view of a flattened embolic coil with distal anchor group with primary and secondary coils, according to an alternate embodiment of the present invention.

FIG. 9A is a detail perspective view illustrating the distal anchor group of an embolic coil, according to an alternate embodiment of the present invention;

FIG. 9B is a detail perspective view illustrating the mandrel portion corresponding to the distal anchor group of an embolic coil, according to an alternate embodiment of the present invention;

FIGS. 10A through 10P illustrate plan views of flattened embolic coils comprising numerous combinations of the distal and near distal coil portions, according to alternate embodiments of the present invention;

FIG. 11A is a front, top, right perspective environmental view illustrating the embolic coil disposed in an aneurysm sac having a constrained opening, according to an alternate embodiment of the present invention;

FIG. 11B is a top perspective view, taken along taken along an equatorial plane (line EP-EP of FIG. 11A), according to an alternate embodiment of the present invention;

FIG. 12A is a side perspective view illustrating the embolic coil disposed in an aneurysm sac having an unconstrained opening, according to an alternate embodiment of the present invention; and

FIG. 12B is a top perspective view, taken along a near normal to an equatorial plane (line EP-EP of FIG. 12A), according to an alternate embodiment of the present invention.

### DESCRIPTION OF THE EMBODIMENTS

Non-limiting embodiments of the present invention will be described below with reference to the accompanying drawings, wherein like reference numerals represent like elements throughout. While the invention has been described in detail with respect to the preferred embodiments thereof, it will be appreciated that upon reading and understanding of the foregoing, certain variations to the preferred embodiments will become apparent, which variations are nonetheless within the spirit and scope of the invention. For a better understanding of the present invention, reference will be made to the following Description of the Embodiments, which is to be read in association with the accompanying drawings, which are incorporated in and constitute a part of this specification, show certain aspects of the subject matter disclosed herein and, together with the description, help explain some of the principles associated with the disclosed implementations.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “some embodiments”, “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawings featured in the figures are provided for the purposes of illustrating some embodiments of the present invention, and are not to be considered as limitation thereto. Term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

### DESCRIPTION OF THE PRIOR ART

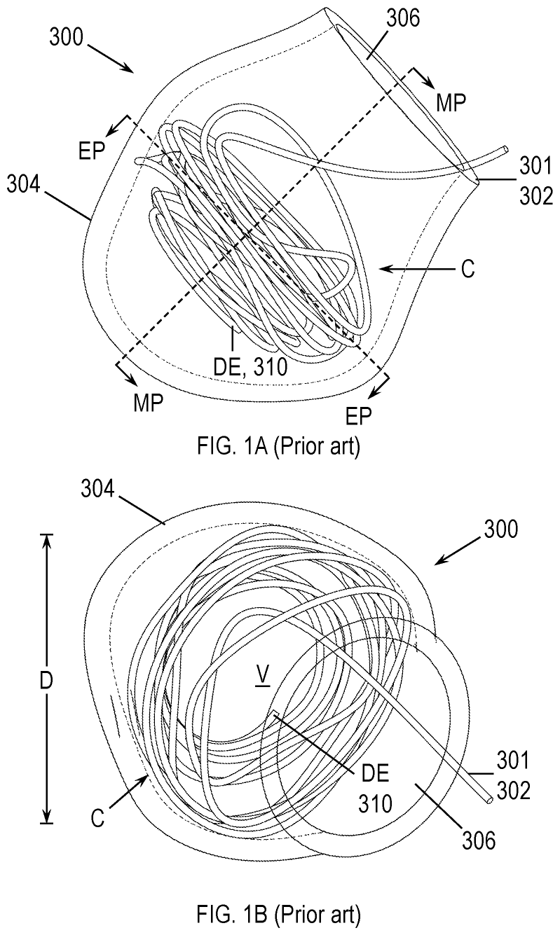

FIGS. 1A-1B illustrate a case where a spherically-wound coil does not engage all surfaces of the inner wall 306 when deposited in an aneurysm sac 300. Contributing factors to this suboptimal deployment may include coil material and length, the shape and diameter of the coil loops, and other design factors. Additional factors may include lower coiling resistance in the middle of the aneurysm, the angle of coil insertion, and/or other anatomical factors. Using x-ray fluoroscopy, spherically-wound conventional embolic coils C can be observed to accumulate along a set of parallel planes or otherwise form groups of coils. These groups of coils tend to arrange in an equatorial plane (EP) region of the aneurysm sac 300 thereby creating one or more voids V open to blood flow that may continue to enter the sac 300. A void V also contributes to a low filling factor, which describes the amount of space occupied by the inserted embolic coil C, thereby requiring numerous additional coils and the associated increased time and cost of the procedure. In FIG. 1A an aneurysm 300 with a constricted opening 302, body 304, and inner wall 306 is filled with a conventional, spherically wound embolic coil C. As the conventional embolic coil C is inserted, a short length of the distal end (DE) may contact the inner wall 306 forming an anchor point 310. At the anchor point 310, the DE of the coil C feels the force of the trailing coil causing it to wind upon itself. The winding action is concentrated in an approximately equatorial plane of diameter D of the aneurysm's irregular shape. In FIG. 1B, the conventional embolic coil C, as viewed from above the equatorial plane EP, has a torus or donut shape leaving a large open space or void V that may be subject to blood pressure and flow and other undesirable factors, thereby preventing the desired clotting.

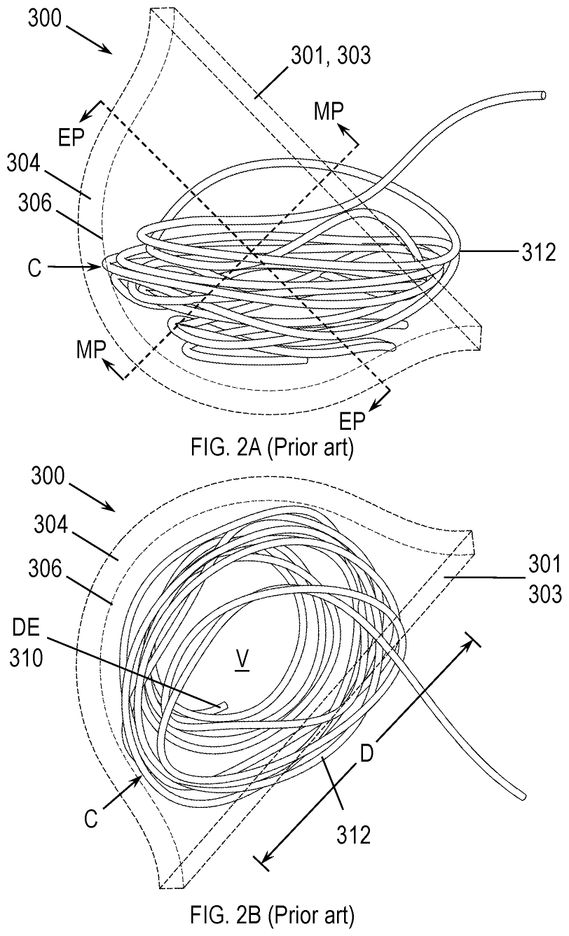

Further disadvantages and problems of conventional coils occur when an aneurysm has an unconstricted opening 303, as shown in FIGS. 2A-2B. As illustrated In FIG. 2A, an aneurysm sac 300 with an unconstricted opening 303, may be characterized by the opening 301 having a larger diameter than the body 304. An unconstricted opening 303 is undesirable as the end(s) of conventional coils C may fail to anchor to the inner wall 306, i.e., to provide a suitable anchor point 310 in an aneurysm sac 300, whereby conventional coils C are susceptible to detachment from the sac 300 representing a serious risk to the patient. Referring to FIG. 2B, a top view taken near normal to the EP of the coiled grouping of FIG. 2A, the conventional coil C winds upon itself thereby producing a toroidal or bird's nest shape thereby creating a void V. The diameter D of the sac and the selected diameter of the conventional coil C may cause the coil to herniate 312 (have a protruding end) and/or dislodge from the aneurysm sac 300 thereby potentially interfering with normal arterial blood flow. As a result of the problems of safe anchorage, framing and fill factor, conventional coils C are not widely used in the treatment of aneurysms with unconstricted openings 303.

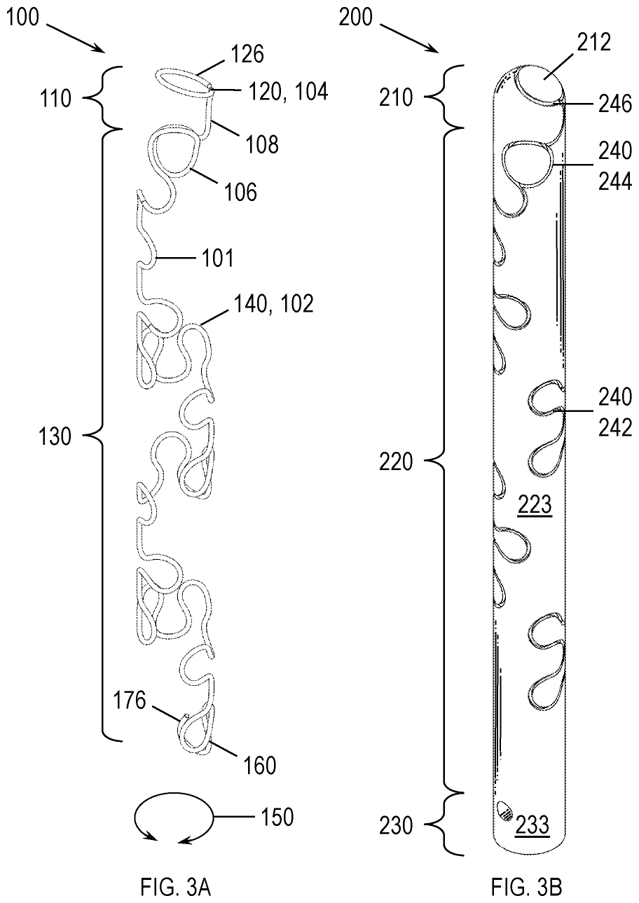

According to an embodiment illustrated in FIGS. 3A-5B an embolic coil 100 may comprise a distal coil portion 110, an intermediate coil portion 130, and a proximal coil portion 160. The embolic coil 100 may be formed a strand of wire material 101, such as, for example, comprising a Heat Set Shapable Wire, Shape Memory Wire and/or other composite materials, metals, and metal alloys selected from the group of consisting of: Gold and/or Gold composites, Platinum and/or Platinum composites including Pt, Pt/Ir, Pt/W, Titanium and/or Titanium composites platinum including Ni/Ti, and other precious metals. Once formed, the embolic coil may be arranged to fit within the catheter of a delivery assembly, as, for example, by straightening the strand of wire 101.

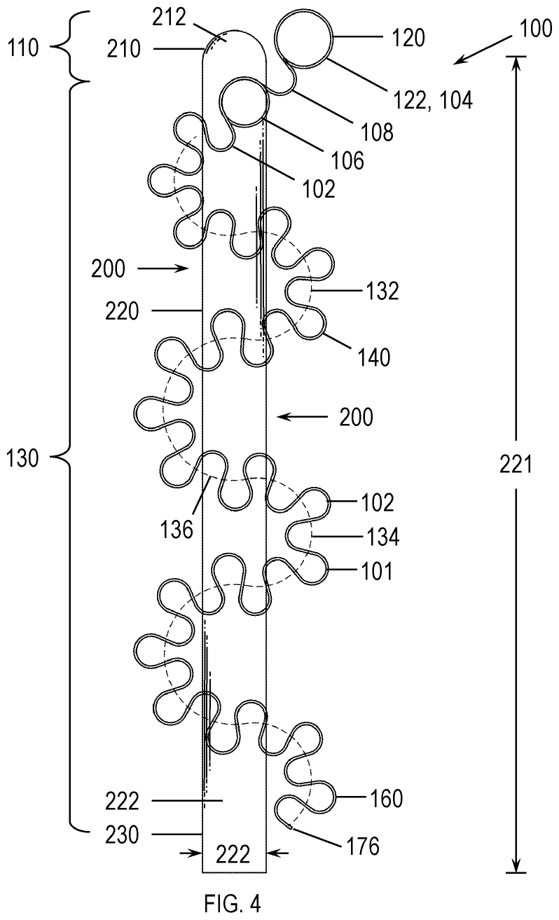

The distal coil portion 110 may comprise an anchor group 120 with a first loop 122 and a second loop 124 which then transitions 108 to an intermediate coil portion 130. First 122 and second 124 loops may be open 102, single- 104 or multi-turn 106 loops. The intermediate coil portion 130 may comprise primary 132, secondary 140 and tertiary 150 coils. Referring to FIG. 4, the primary coil portion 130 may comprise a primary coil loop 132 of an extended length, which may be configured with a predetermined set of arcuate curves 134, 136, for example S-turns, omega-shaped turns or semi-circular turns. The arcuate curves may be of varying radius of curvature, such as, for example, a first radius 134 and a second radius 136. The curves 134, 136 may be arranged in concatenated or staggered fashion. The arcuate curves 134, 136 may be formed on a cylindrical portion 220 of the mandrel 200 having a length 221 and diameter 222 that may be varied in dimensions to match the size of the embolic coil 100 to the opening 301 and/or body 304 of the aneurysm sac 300. Superimposed on the primary coil loop 132 may be a secondary coil 140, also comprising a predetermined set of arcuate curves. The set of curves of the secondary coil 140 may comprise open 102, multi-turn 106, and transition 108 loops. The proximal coil portion 160 of the embolic coil 100 may terminate in an open end 176.

Referring to FIG. 3B, the wire 101 may be formed into a coil by winding it around a groove or channel 240 formed in the spherical and cylindrical surface(s) of the mandrel 200. The mandrel 200 may comprise a distal portion 210 with a spherical surface 212, an intermediate portion 220 with a cylindrical surface 223, and proximal portion 230 with a cylindrical surface 233. The mandrel may further comprise a groove 240 which holds the wire 101 on the mandrel 200 during fabrication. The groove 240 may be formed by computer numeric controlled machining, milling, laser machining, casting, additive manufacturing or other suitable technique. The depth of the groove 240 may vary depending on whether the specific section of the groove 240 is intended to hold an open 102, single-turn 104, multi-turn 104, or transition 108 loop of the wire 101. For example, the groove 240 may have a single-wire depth 242 for forming open 102, single-turn 104, or transition 108 loops or a multi-wire depth 244 for forming multi-turn 106 loops. In this way, the primary 130 and secondary 140 coil portion shapes are imparted to the coil 100 by the groove 240, as illustrated in FIGS. 3A-3B. In addition, the wrapping of the wire 101 around the mandrel 200 results in the embolic coil 100 having a tertiary coil portion 150 such as, for example, a c-shaped coil, as shown in FIG. 3A. In this way, the device may be considered to have the varying arcuate shapes of the primary 132, secondary 140, and tertiary 150 coil portions formed by the groove 240 cut into a combination of the spherical 212 and cylindrical 223 surfaces of the mandrel 200.

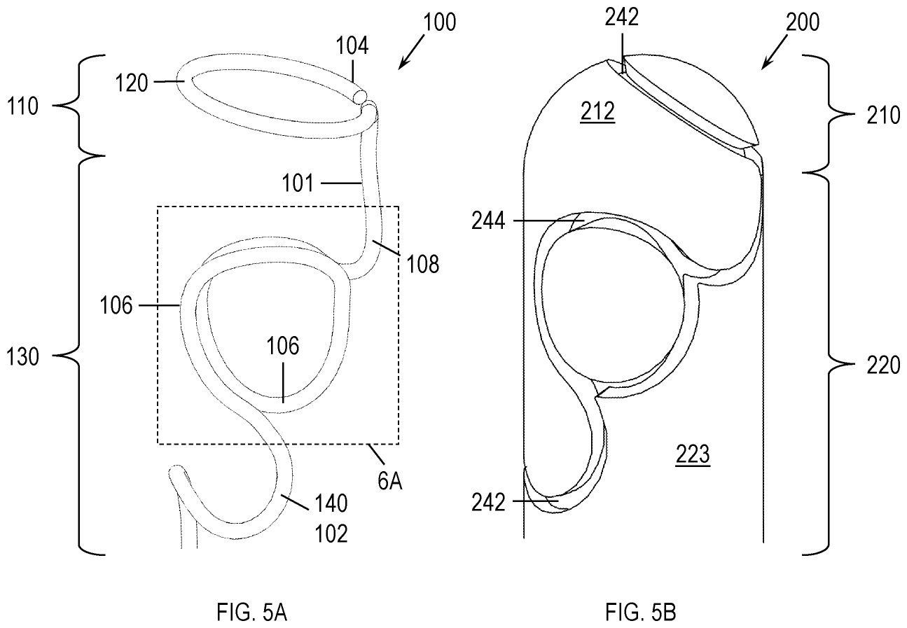

Referring to FIGS. 5A-5B, the embolic coil 100 may be characterized by a distal coil portion 110 comprising a first loop 122 forming an anchor 120. The term “anchor” applies in two scenarios: in manufacturing it refers to the securing of the first end of the wire 101 to the mandrel groove 240, whereas in deployment it refers to the fixing of the distal end 110 of the embolic coil 100 to the wall of the aneurysm. The distal coil 110 may adjoin an intermediate coil portion 130 comprising a transition loop 108, a multi-turn loop 106 and other secondary loops 140. The first loop 122 may be an arc of less than 360° thereby leaving a small gap, as depicted in FIG. 5A. The gap may comprise an arc of, for example, less than about 20°. FIG. 5B shows a closeup of the distal 210 and intermediate 220 portions of the mandrel 200 upon which the wire 101 is shaped. The anchor 120 may be secured to the single-wire depth groove 242 in the spherical surface 212 and aid the manufacturing process by securing a first end of the wire 101 to the mandrel 200. Sufficient tension may then be applied to the wire 101 to place it into the groove 240 without slack and further providing friction between the wire 101 and groove 240 to aid retention. At the multi-turn loop 106 portion of the groove 240, which lies on the cylindrical surface 223 of the mandrel 200, the groove 240 may have a depth 244 that is slightly greater than twice the diameter of the wire 101, thereby allowing it to be once overlaid. A smooth transition from single- 242 to multi-wire 244 depth and vice versa may occur in the portions of the groove 240 corresponding to the transition portions 108 of the coil 100.

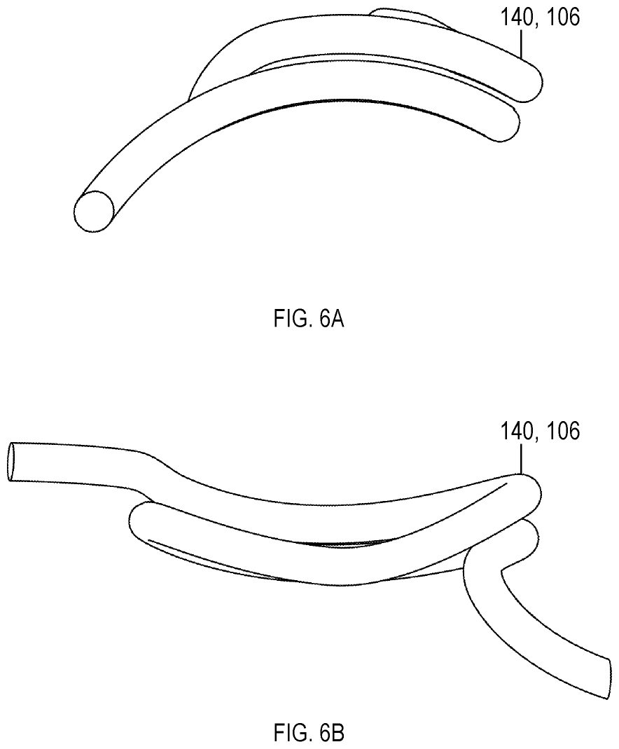

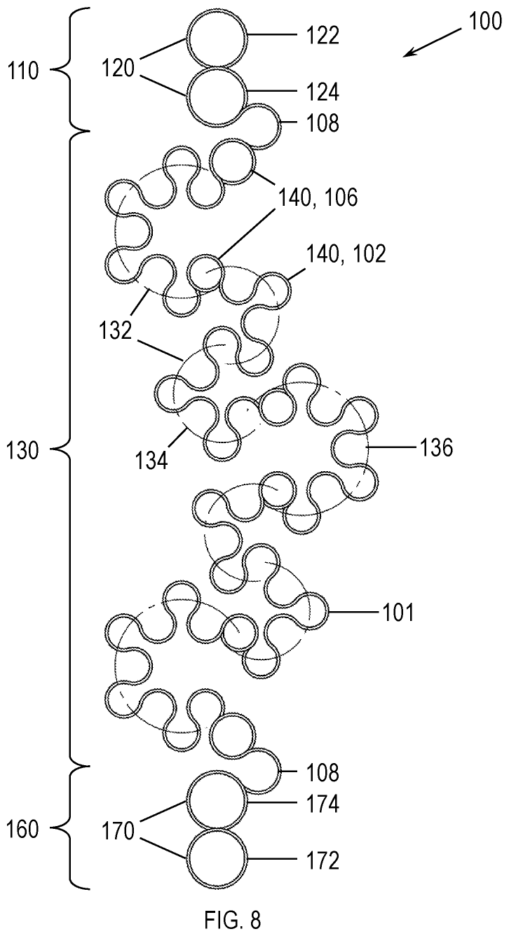

FIGS. 6A-6B illustrate additional details of the loop structure of a secondary coil 140. All secondary coils 140 bend about a primary axis perpendicular to the surface upon which they are formed, the primary axis located at the center of curvature of the loop, as shown in FIGS. 4 and 8. Secondary coils 140 may have one or two additional axes of curvature. For example, FIG. 6A shows a multi-turn loop 106 from the intermediate coil 130 as viewed from the bottom of the embolic coil 100, and/or proximal portion 160, where shown by a generally convex shape with an axis of curvature parallel to the axis of the mandrel 200, normal to the plane of the image, below the loop, and/or coincident. FIG. 6B shows the same multi-turn loop 106 as viewed from the side where it can be seen that it has a generally concave shape with an axis of curvature perpendicular to the axis of the mandrel, as illustrated normal to the plane of the image, and located at an offset distance therefrom as shown above the loop. This type of bend is imparted to the loop by varying the depth of the groove 240. All secondary coils may comprise the former type of bend, while some, specifically anchor loops 122, 124, 172, 176 and multi-turn loops 106 may also comprise the latter type of bend. Loops with both convex and concave bends may aid the conformal anchoring and framing of the undulating inner wall 306 of an aneurysm sac 300.

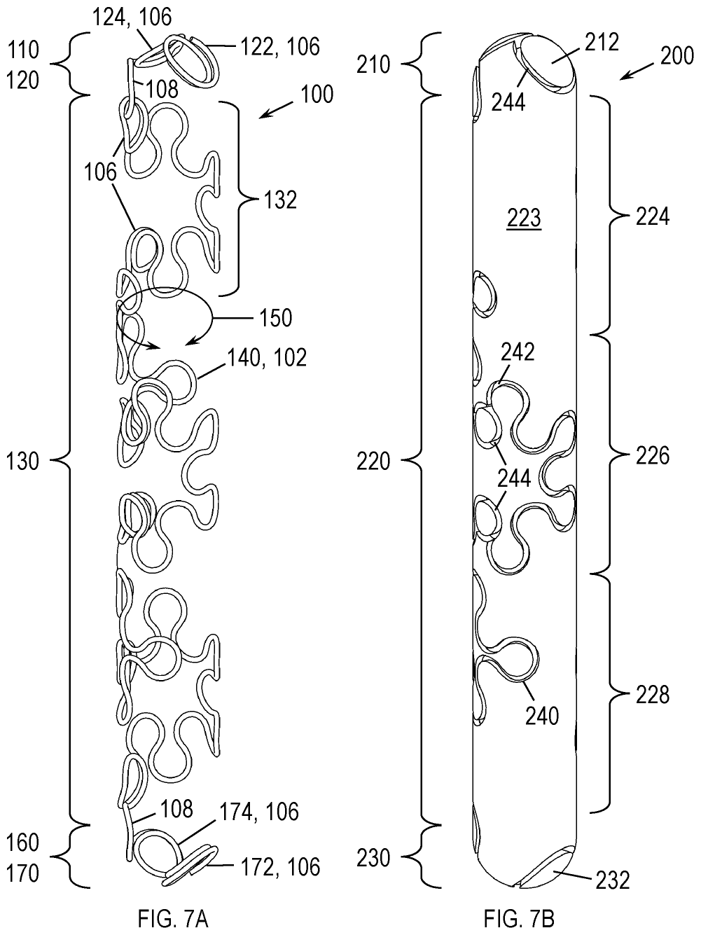

FIGS. 7A-7B illustrate an alternate embodiment of the embolic coil 100 comprising a distal coil portion 110, an intermediate coil portion 130, and a proximal coil portion 160. The embolic coil 100 may be formed from the strand of wire 101, such as, for example, a heat set shapable wire, shape memory wire, and/or other composite materials, metals, and metal alloys selected from the group of consisting of: Gold and/or Gold composites, Platinum and/or Platinum composites including Pt, Pt/Ir, Pt/W, Titanium and/or Titanium composites platinum including Ni/Ti, and other precious metals. Once formed, the embolic coil may be arranged to fit within the catheter of a delivery assembly, as, for example, by straightening the strand of wire 101.

The distal coil portion 110 may comprise an anchor group 120 with a first loop 122 and a second loop 124. First 122 and second 124 loops may be open 102, single- 104 or multi-turn 106 loops. An intermediate coil portion 130 may be coupled to the distal coil portion 110 and comprises primary 132, secondary 140 and tertiary 150 coils. Referring to FIG. 8, the primary coil portion 130 may comprise a primary coil loop 132 of an extended length, which may be configured with a predetermined set of arcuate curves 134, 136, for example S-turns, omega-shaped turns or semi-circular turns. The arcuate curves may be of varying radius of curvature, such as, for example, a first radius 134 and a second radius 136. Superimposed on the primary coil loop 132 may be a secondary coil 140, also comprising a predetermined set of arcuate curves. The set of curves of the secondary coil 140 may comprise open 102, multi-turn 106, and transition 108 loops. The proximal end 160 of the embolic coil 100 comprises an anchor group 170 with a first loop 172 and a second loop 174. First 172 and second 174 loops may be single- 104 or multi-turn 106, loops, as shown in FIG. 7A.

Referring to FIG. 7B, the wire may be formed into a coil by winding it around a mandrel 200. The mandrel 200 may comprise a distal portion 210 with a spherical surface 212, an intermediate portion 220 with a cylindrical surface 223, and a proximal portion 230 with a spherical surface 232. The intermediate portion 220 may further comprise distal 224, middle 226 and proximal 228 portions. The mandrel 200 may further comprise a groove 240 which may retain the wire on the mandrel 200 during fabrication. The depth of the groove 240 may vary depending on whether the specific section of the groove 240 is intended to hold an open 102, single-turn 104, multi-turn 104, or transition 108 loop of the wire 101. Thus, the primary 130 and secondary 140 coil portion shapes may be imparted to the coil 100 by the groove 240, as illustrated in FIGS. 7A-7B. In addition, the wrapping of the wire 101 around the mandrel 200 may result in the embolic coil 100 having a tertiary coil portion 150 such as, for example, a c-shaped coil, as shown in FIG. 7A. In this way, the device may be considered to have the varying arcuate shapes of the primary 132, secondary 140, and tertiary 150 coil portions formed by the groove 240 cut into a combination of the spherical 212, 232 and cylindrical 223 surfaces of the mandrel 200.

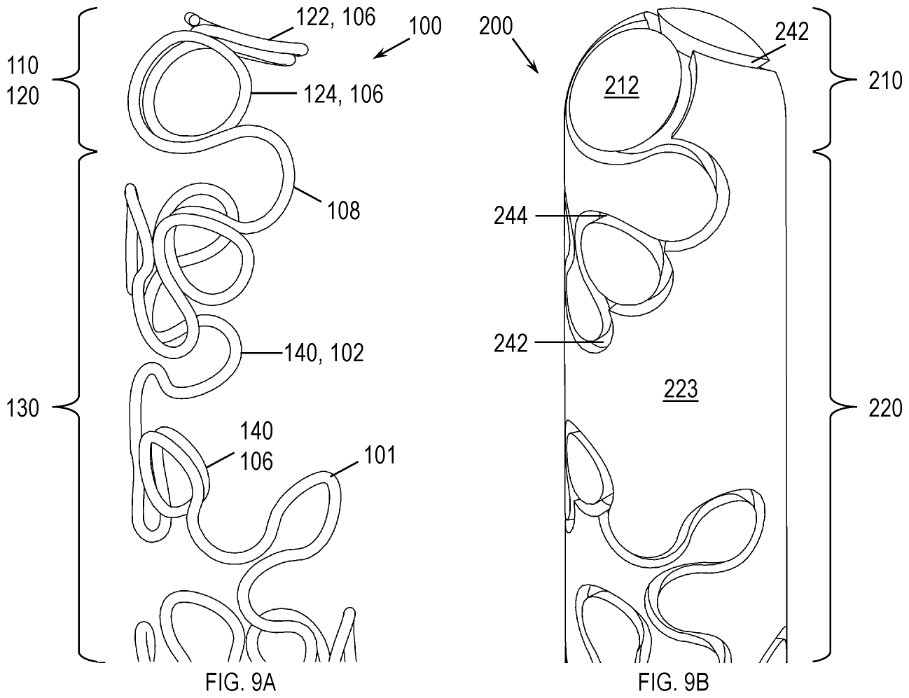

Referring to FIGS. 9A-9B, the embolic coil may be characterized by a distal end 110 comprising first 122 and second 124 loops 122 forming an anchor group 120. The distal coil 110 may adjoin an intermediate coil portion 130 comprising a transition loop 108, a multi-turn loop 106 and other secondary loops 140. The first loop 122 may comprise a multi-turn loop 106 having an arc of approximately 720° thereby overlapping itself completely. Likewise, the second loop 124 may comprise a single- 104 or multi-turn loop 106. FIG. 9B shows a detail view of the distal end 210 and proximal intermediate portion 220 of the mandrel 200 upon which the wire 101 is formed. The anchor group 120 may be secured to a single-wire depth groove 242 in the spherical surface 212 and aids the manufacturing process by securing a first end of the wire 101 to the mandrel 200. Sufficient tension may then be applied to the wire 101 to place it into the groove 240 without slack and further providing friction between the wire 101 and groove 240 to aid retention. At the multi-turn loop 106 portion of the groove 240, which lies on the cylindrical surface 223 of the mandrel 200, the groove 240 may have a depth 244 that is slightly greater than twice the diameter of the wire 101, thereby allowing it to be once overlaid. A smooth transition from single- 242 to multi-wire 244 depth and vice versa may occur in the portions of the groove 240 corresponding to the transition portions 108 of the coil 100.

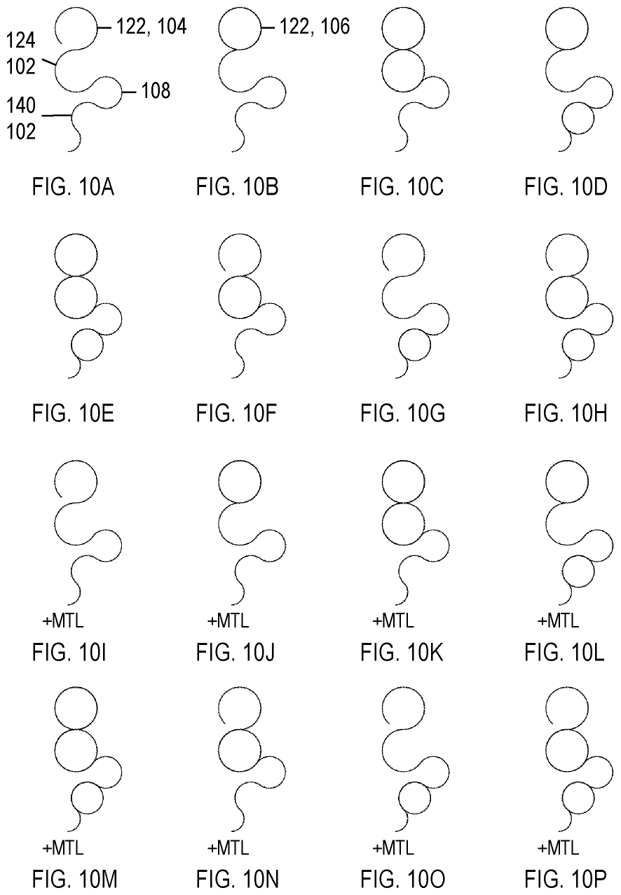

As previously mentioned, the first few loops of the distal and intermediate coils may provide an anchoring function both in the manufacturing process and in the deployment process. Therefore, the number and nature of the loops may have a large effect on the success of the coil as a product. For example, in general, closed (single- or multi-turn) loops may provide better anchoring than open loops and may do so at relatively lower pressure against the aneurysm wall. In manufacturing, multi-turn may be more robust anchors than single-turn loops. In contrast, open loops may be more flexible than closed loops and may provide deployment advantages, such as promoting non-parallel plane folding and/or deformation. Thus, various combinations and permutations of the near-terminal (distal and proximal portions) and intermediate loops may be used to tailor the performance of the coil for both manufacturing and use cases. FIGS. 10A-10P illustrate the various near-terminal combinations of three loops: a first distal loop 122, a second distal loop 124 and a first intermediate loop 140, with a transition 108 between the distal 122, 124 and intermediate 140 loops. For clarity, elements are labeled in FIGS. 10A and 10B only. In FIGS. 10A-10P, open loops 102 are indicated by a semi-circular or omega shape, single-turn loops 104 are indicated by nearly closed circles and multi-turn loops 106 are indicated by full circles. FIGS. 10A-10H represent permutations attached to an intermediate coil that comprises strictly open loops 102 (not shown), while FIGS. 101-10P represent the same permutations attached to an intermediate coil with both open 102 and multi-turn loops (MTLs) 106 (also not shown), as illustrated in FIG. 8. In both cases, the mirror image of the anchor group 120 sequence may appear at the proximal end (not shown) of the embolic coil 100. In FIG. 10A a first 122, single-turn 104 (i.e near complete) loop may adjoin a second 124, open 102 loop, followed by a transition loop 108 and a first intermediate open loop 102. In FIG. 10B, a first, multi-turn loop 106 (indicated by the closed circle) is adjoined by a second, open loop 102, followed by a transition loop 108 and a first intermediate open loop 102, as before. In FIG. 10C, first and second loops are double loops, while the following transition and first intermediate loops are open. The remaining permutations of FIGS. 10D-10H may be interpreted in a similar way. In summary, a wide variety of loop permutations are contemplated herein leading to differences in coil performance that may be selected for reasons of manufacturability, deployment, or patient specific needs.

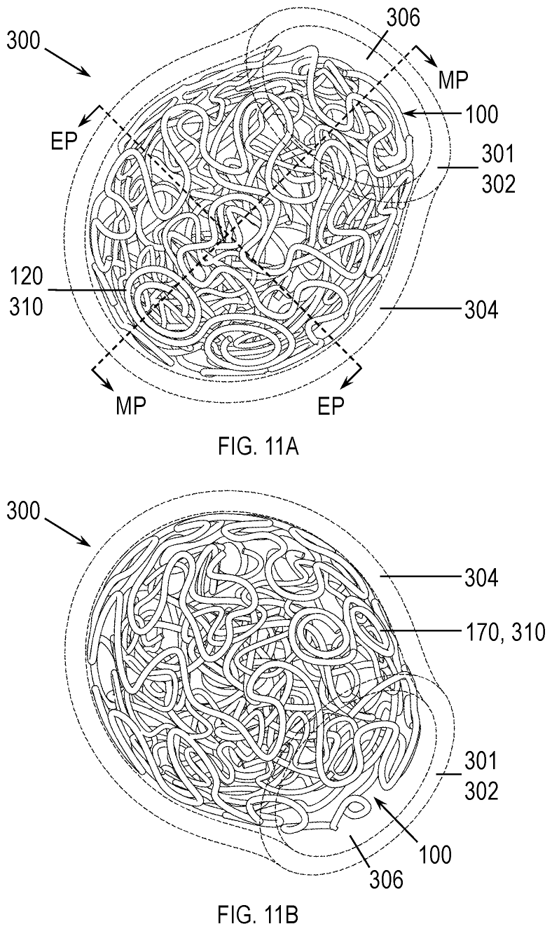

The strand of wire 101 may be wound around the mandrel 200 in a predetermined pattern defined by the groove 240, as shown in FIGS. 3A-9B. A heat treatment may be applied to set the shape memory of the wire 101. At room temperature, the wire 101 may then be straightened to be inserted into a delivery catheter. At the aneurysm site, as the wire 101 is ejected from the catheter, it immediately recovers the predetermined, coiled shape as it enters the aneurysm 300. A coil 100 deployed within an aneurysm sac 300 will self-curl roughly defining the surface of a sphere. Because the primary 132, secondary 140 and tertiary 150 coil portions can flex, the embolic coil 100 arrangements may be compressed. The overall geometry of the device is also highly flexible, with various sized loops, and can conform to a wide variation in aneurysm size and shape, including and in particular, to the size of the opening 301 of the aneurysm. FIGS. 11A-11B illustrate the deployed coil in an aneurysm 300 with a constricted opening 302. Referring to FIG. 11A, the distal portion of the coil with the anchor group 120 may contact the inner wall 306 of the aneurysm 300 first and establishes an anchor point 310. Advantageously, the loops of the anchor group 120 may contact the body of the aneurysm 304 over a large area relative to the open end of the spherically wound coil C of FIGS. 1A-1B. This may reduce the pressure on the inner wall 306 thereby reducing the risk of damage or rupture. The primary 130 and secondary 140 loops of the coil may serve to cause coalescing of the coil 100 onto random, non-parallel planes. In this way, the coil “seeks out” empty space to occupy within the aneurysm body 304. Referring to FIG. 11B, the anchor group 170 of the proximal end of the coil 100 may form another anchor point 310 within the aneurysm body 304 thereby forming a very stable structure completely confined within the body 304 and opening 301 and reducing the risk of coil herniation 312 as illustrated in FIGS. 2A-2B. The coil 100 has a very high fill factor compared to a spherically wound coil C as illustrated in FIGS. 1A-1B.

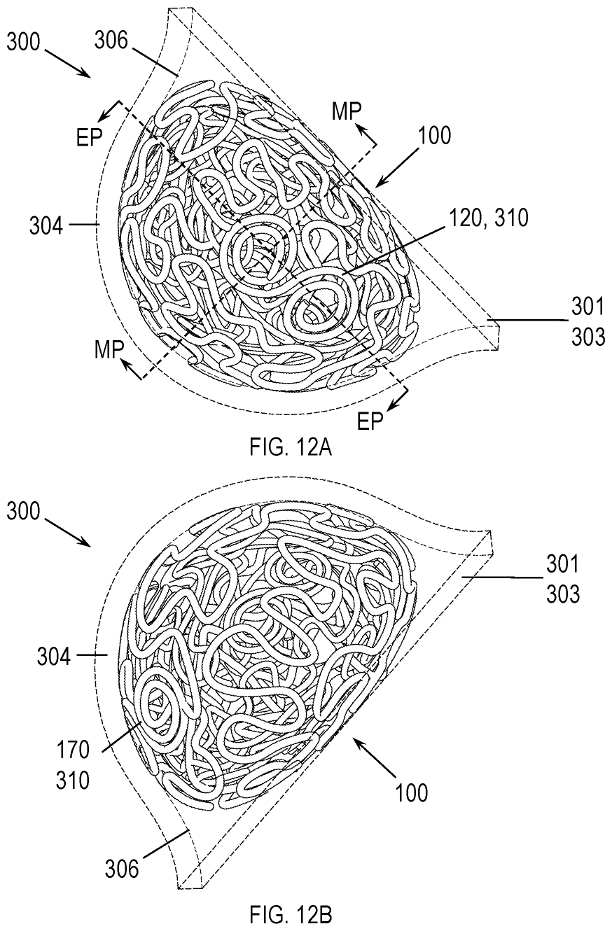

The embolic coil 100 of the present disclosure may have advantages for use in more challenging aneurysm cases. FIGS. 12A-12B illustrate the deployed coil in an aneurysm 300 with an unconstricted opening 303. Referring to FIG. 12A, the anchor group 120 of the distal portion 110 may contact the inner wall 306 of the aneurysm 300 first and establishes an anchor point 310. Advantageously, the loops of the anchor group 120 contact the body of the aneurysm 304 over a large area relative to the spherically wound coil C of FIGS. 2A-2B. This may reduce the pressure on the inner wall 306 thereby reducing the risk of damage or rupture. The primary 130 and secondary 140 loops of the coil may serve to cause coalescing of the coil 100 onto random, non-parallel planes. In this way, the coil “seeks out” empty space to occupy within the aneurysm body 304. Referring to FIG. 12B, the anchor loop group 170 of the proximal end of the coil may form another anchor point 310 within the aneurysm body 304 thereby forming a very stable structure completely confined within the body 302 and reducing the risk of coil herniation 312 as illustrated in FIGS. 2A-2B. The coil 100 may have a very high fill factor compared to a spherically wound coil as illustrated in C FIGS. 1A-1B.

The previous description of the disclosed embodiments is provided to enable any person skilled in the art to make or use the invention. Various modifications to these embodiments will be readily apparent to those skilled in the art, and the generic principles defined herein can be applied to other embodiments without departing from the spirit or scope of the invention. For example, materials other than metal, such as polymer, may be used for the coil. It is therefore desired that the present embodiments be considered in all respects as illustrative and not restrictive, reference being made to the appended claims as well as the foregoing descriptions to indicate the scope of the invention.

Element List:

| Number | Element |
|---|---|
| C | Conventional coil |
| DE | Distal end |
| EP | Equatorial plane |
| D | Diameter |
| V | Void |
| 100 | Embolic coil |
| 101 | Strand of wire |
| 102 | Open loop |
| 104 | Single turn loop |
| 106 | Multiple or multi-turn loop |
| 108 | Transition loop |
| 110 | Distal coil portion |
| 120 | Anchor group |
| 122 | First loop D1 |
| 124 | Second loop D2 |
| 130 | Intermediate coil portion |
| 132 | Primary coil portion |
| 134 | Primary coil first radius |
| 136 | Primary coil second radius |
| 140 | Secondary coil portion |
| 150 | Tertiary coil portion (C-shape) |
| 160 | Proximal coil |
| 170 | Anchor group |
| 172 | First loop D1 |
| 174 | Second loop D2 |
| 176 | Open termination |
| 200 | Mandrel |
| 210 | Distal portion |
| 212 | Spherical surface |
| 220 | Intermediate portion |
| 221 | Length |
| 222 | Diameter |
| 223 | Cylindrical surface |
| 224 | Distal portion |
| 226 | Middle portion |
| 228 | Proximal portion |
| 230 | Proximal portion |
| 232 | Spherical surface |
| 233 | Cylindrical surface |
| 240 | Groove or channel |
| 242 | Single-wire depth |
| 244 | Multi-wire depth |
| 300 | Aneurysm sac |
| 301 | Opening |
| 302 | Constricted |
| 303 | Unconstricted |
| 304 | Body |
| 306 | Inner wall |
| 308 | Protruding end |
| 310 | Anchor point |
| 312 | Herniated coil |

What is claimed is:

1. An embolic coil of a unitary a strand of wire material, the embolic coil comprising:
a distal coil portion, a proximate coil portion, and an intermediate coil portion extending between said distal and proximate coil portions,
said distal coil portion having an anchor loop formed from an open-loop unit, said open-loop unit operably connecting to a distal end transition loop unit formed between said distal coil portion and said intermediate coil portion,
said intermediate coil portion comprising:
a primary coil portion flexibly connected to said transition coil portion, said primary coil comprising a predetermined set of arcuate curves having a first radius and a second radius,
a secondary coil portion comprising a plurality of arcuate curves having radii smaller than said first and second radii of said primary coil portion and that follow the trajectory of the primary coil, and
said proximate coil portion having a delivery loop formed from an open-loop unit, said open-loop unit operably connecting to a proximal end transition loop unit thereby operably connecting said primary coil portion and/or said secondary coil portion that occurs between said proximate coil portion and said intermediate coil portion,
whereby said anchor loop operable to secure to an inner surface of a vessel at a site of an embolization procedure.

2. The embolic coil of claim 1 wherein said anchor loop being formed on a plane on a spherical tip of a mandrel with at least one loop unit selected from the group consisting of (i) an open-loop unit, (ii) a single-loop unit, and (iii) a multi-turn loop unit.

3. The embolic coil of claim 1 wherein said transition loop is at least one loop unit selected from the group consisting of (i) said open-loop unit, (ii) said single-loop unit, (iii) said multi-turn loop unit, (iv) said primary-loop unit, (v) said secondary-loop unit, and (vi) said transition loop unit.

4. The embolic coil of claim 1 wherein said distal end transition loop unit operably connects said intermediate coil portion and said distal coil portion using at least one loop unit selected from the group consisting of (i) said open-loop unit, (ii) said single-loop unit, (iii) said multi-turn loop unit, (iv) said primary-loop unit, (v) said secondary-loop unit, and (vi) a transition loop unit.

5. The embolic coil of claim 1 wherein the arcuate curves of the primary coil portion include one or more one loop unit selected from the group consisting of: (i) said open-loop unit, (ii) said single-loop unit, (iii) said multi-turn loop unit, and (iv) a transition loop unit.

6. The embolic coil of claim 1 wherein the arcuate curves of the secondary coil portion include one or more one loop unit selected from the group consisting of: (i) said open-loop unit, (ii) said single-loop unit, (iii) said multi-turn loop unit, and (iv) said transition loop unit.

7. The embolic coil of claim 1 wherein said proximal end transition loop unit operably connects to said delivery coil loop between said intermediate coil portion and said proximal end portion using at least one loop unit selected from the group consisting of (i) said open-loop unit, said single-loop unit, (iii) said multi-turn loop unit, (iv) said primary-loop unit, (v) said secondary-loop unit, and (vi) a transition loop unit.

8. The embolic coil of claim 1 wherein the strand of wire material comprises a Heat Set Shapable Wire, Shape Memory Wire and/or other composite materials, metals, and metal alloys selected from the group of consisting of: Gold and/or Gold composites, Platinum and/or Platinum composites including Pt, Pt/Ir, Pt/W, Titanium and/or Titanium composites platinum including Ni/Ti, and other precious metals.

9. An embolic coil of a strand of wire material, said embolic coil comprising:
a distal coil portion, a proximate coil portion, and an intermediate coil portion extending between said distal and proximate coil portions,
said distal coil portion comprising:
an anchor loop with at least one loop unit selected from the group consisting of (i) an open-loop unit, (ii) a single-loop unit, (iii) a multi-turn loop unit, said anchor loop configured to transition to at least one loop unit selected from the group consisting of (i) said open-loop unit, said single-loop unit, (iii) said multi-turn loop unit, (iv) a primary-loop unit, (v) a secondary-loop unit, and (vi) a transition loop unit;
said intermediate coil portion comprising:
a transition coil portion flexibly connected to said distal coil portion and/or proximal coil portions,
a primary coil portion flexibly connected to said transition coil portion, said primary coil comprising a predetermined set of arcuate curves having a first radius and a second radius,
a secondary coil portion comprising a plurality of arcuate curves having radii smaller than said first and second radii of said primary coil portion and that follow the trajectory of the primary coil; and
said proximate coil portion comprises:
at least one delivery loop selected from the group consisting of (i) said open-loop unit, (ii) said single-loop unit, (iii) said multi-turn loop unit, and (iv) said transition loop unit,
whereby said anchor loop maintains a position on an inner surface of a vessel at a site of an embolization procedure and the embolic coil, in a relaxed state in the patient's body, substantially assumes the shape of the inner surface of the vessel at the site of an embolization procedure.

10. The embolic coil of claim 9 wherein the arcuate curves of the primary coil portion includes one or more one loop unit selected from the group consisting of: (i) said open-loop unit, (ii) said single-loop unit, (iii) said multi-turn loop unit, and (iv) said transition loop unit.

11. The embolic coil of claim 9 wherein the arcuate curves of the secondary coil portion includes one or more one loop unit selected from the group consisting of: (i) said open-loop unit, (ii) said single-loop unit, (iii) said multi-turn loop unit, and (iv) said transition loop unit.

12. The embolic coil of claim 9 wherein the strand of wire material comprises a Heat Set Shapable Wire, Shape Memory Wire and/or other composite materials, metals, and metal alloys selected from the group of consisting of: Gold and/or Gold composites, Platinum and/or Platinum composites including Pt, Pt/Ir, Pt/W, Titanium and/or Titanium composites platinum including Ni/Ti, and other precious metals.

13. An embolic coil of a unitary wire strand, said embolic coil comprising:
a distal coil portion, a proximate coil portion, and an intermediate coil portion extending between said distal and proximate coil portions, said distal coil portion having an anchor loop with at least one loop unit selected from the group consisting of:
(i) a single-loop unit, and
(ii) a multi-turn loop unit, a transition coil portion flexibly connected to said anchor loop between said distal coil portion and said intermediate coil portion, said intermediate coil portion has a primary coil portion flexibly connected to said transition coil portion, said primary coil comprising a predetermined set of arcuate curves having a first radius and a second radius, a secondary coil portion comprising a plurality of arcuate curves having radii smaller than said first and second radii of said primary coil portion and that follow the trajectory of the primary coil, and said proximate coil portion having a delivery loop selected from the group consisting of:
a) said open-loop unit,
b) said single-loop unit, and
c) said multi-turn loop unit, whereby said anchor loop maintains a position on an inner surface of a vessel at a site of an embolization procedure and the embolic coil, in a relaxed state in the patient's body, substantially assumes the shape of the inner surface of the vessel at the site of an embolization procedure.

14. An embolic coil of a unitary a strand of wire material, said embolic coil comprising:
a distal coil portion, a proximate coil portion, and an intermediate coil portion extending between said distal and proximate coil portions,
said distal coil portion comprising: an anchor loop with at least one loop unit selected from the group consisting of (i) an open-loop unit, (ii) a single-loop unit, (iii) a multi-turn loop unit, said anchor loop flexibly coupled to a transition open-loop formed on a second plane on a spherical tip of a mandrel and forming an arc of between about 180° and about 520°;
said intermediate coil portion comprising a primary coil flexibly coupled to said transition open-loop, said primary coil comprising a predetermined set of arcuate curves having a first radius and a second radius loop formed on a plurality of planes on a cylindrical portion of said mandrel, a secondary coil comprising a plurality of arcuate curves having radii smaller than the first and second radii and that follow the trajectory of the primary coil formed on a plurality of planes on a cylindrical portion of said mandrel; and
said proximate coil portion comprising a delivery loop formed by an open-loop unit connected to a proximal end transition loop unit, said proximal end transition loop unit formed by an open-loop unit flexibly connected to said primary coil portion and/or said secondary coil portion that occurs between said intermediate coil portion and said proximate coil portion.

15. The embolic coil of claim 14 wherein said anchor loop is selected from at least one loop unit selected from the group consisting of (i) an open-loop unit, (ii) a single-loop unit, and (iii) a multi-turn loop unit.

16. The embolic coil of claim 14 wherein said transition loop is selected from at least one loop unit selected from the group consisting of (i) said open-loop unit, (ii) said single-loop unit, (iii) said multi-turn loop unit, (iv) said primary-loop unit, (v) said secondary-loop unit, and (vi) said transition loop unit.

17. The embolic coil of claim 14 wherein said distal end transition loop unit operably connects said intermediate coil portion and said distal coil portion using at least one loop unit selected from the group consisting of (i) said open-loop unit, (ii) said single-loop unit, (iii) said multi-turn loop unit, (iv) said primary-loop unit, (v) said secondary-loop unit, and (vi) a transition loop unit.

18. The embolic coil of claim 14 wherein the arcuate curves of the primary coil portion include one or more one loop unit selected from the group consisting of: (i) said open-loop unit, (ii) said single-loop unit, (iii) said multi-turn loop unit, and (iv) a transition loop unit.

19. The embolic coil of claim 14 wherein the arcuate curves of the secondary coil portion include one or more one loop unit selected from the group consisting of: (i) said open-loop unit, (ii) said single-loop unit, (iii) said multi-turn loop unit, and (iv) said transition loop unit.

20. The embolic coil of claim 14 wherein said proximal end transition loop unit operably connects to said delivery coil loop between said intermediate coil portion and said proximal end portion using at least one loop unit selected from the group consisting of (i) said open-loop unit, said single-loop unit, (iii) said multi-turn loop unit, (iv) said primary-loop unit, (v) said secondary-loop unit, and (vi) a transition loop unit.

21. The embolic coil of claim 14 wherein the strand of wire material comprises a Heat Set Shapable Wire, Shape Memory Wire and/or other composite materials, metals, and metal alloys selected from the group of consisting of: Gold and/or Gold composites, Platinum and/or Platinum composites including Pt, Pt/Ir, Pt/W, Titanium and/or Titanium composites platinum including Ni/Ti, and other precious metals.
